(19) United States
(12) Patent Application Publication
(10) Pub. No.: US 2017/0071866 A1
(43) Pub. Date: Mar. 16, 2017

(54) NANOPARTICLES LOADED WITH ACTIVE INGREDIENTS, THEIR PROCESS OF PREPARATION, AND THEIR USES

(71) Applicant: Onxeo S.A., Paris (FR)

(72) Inventor: Christelle Zandanel, Fontenay Aux Roses (FR)

(21) Appl. No.: 15/265,247

(22) Filed: Sep. 14, 2016

Related U.S. Application Data
(60) Provisional application No. 62/218,293, filed on Sep. 14, 2015.

(51) Int. Cl. A61K 9/51; A61K 31/704

(52) U.S. Cl. CPC A61K 9/5123; A61K 9/5138; A61K 9/5161; A61K 31/704

(57) ABSTRACT

The present disclosure concerns nanoparticles loaded with active ingredients with controlled average diameter, their processes of preparation, their formulations, and their therapeutic uses.

### FIELD OF THE DISCLOSURE

The present disclosure concerns the field of pharmaceuticals, in particular the field of pharmaceutical nanoparticles and, more specifically, nanoparticles loaded with active ingredients.

### BACKGROUND

WO99/43359 describes nanoparticles loaded with active ingredients such as chemotherapeutic agents. The nanoparticles are prepared by mixing (i) an active ingredient with (ii) a complexing agent, such as a cyclodextrin, and (iii) a monomer, such as an alkylcyanoacrylate monomer. While WO99/43359 notes that a surfactant or stabilizing agent, such as dextran or a poloxamer, can be used in the preparation of the nanoparticles, WO99/43359 also notes that such agents are not necessary because said cyclodextrins have a sufficient stabilizing effect on the nanoparticles for the surfactant agent to be omitted. The polymerization of said monomer is then conducted to obtain nanoparticles loaded with the active ingredient. According to WO99/43359, the complexing agent complexes the active ingredient during preparation of the nanoparticle so as to protect it against chemical reactions occurring during the formation of the nanoparticle. WO99/43359 and its US counterparts (e.g., U.S. Pat. No. 6,881,421) are incorporated by reference herein in their entirety.

WO2012/131018 discloses the use of said nanoparticles of WO99/43359 loaded with doxorubicin for treating cancer, by intravenous or intra-arterial infusion, for at least 2 hours. These nanoparticles loaded with doxorubicin are currently developed under the trademark name Livatag® for the treatment of Hepatocellular Carcinoma (HCC) in particular. WO2012/131018 and its US counterparts (e.g., U.S. Published Patent Application No. 2014/0024610) are incorporated by reference herein in their entirety.

HCC is known as hypervascular solid liver cancer and is characterized by a high degree of drug resistance. The mechanisms of this chemoresistance in HCC are multiple. The most common mechanism is related to the multidrug resistance (MDR) transporters or pumps known as permeability glycoprotein (P-gp) and multidrug resistance-associated protein (MRP). These transporters or pumps allow tumor cells to efflux different types of chemotherapeutic agents into the extracellular environment.

However, said nanoparticles of WO99/43359 display unique mechanisms to bypass multi-drug resistance that can be summarized as follows. The nanoparticles loaded with doxorubicin adsorb to the surface of tumor cells and release the entrapped doxorubicin close to the cell membrane which leads to a high local gradient concentration. The nanoparticles then degrade and release soluble polycyanoacrylic acid which might interact with the plasma membrane and contribute to improve the intracellular delivery of doxorubicin. The soluble polycyanoacrylic acid could also mask the positive charge of doxorubicin thus preventing its efflux by the P-gp or MRP pumps.

The nanoparticles are thus believed to fight against chemotherapy resistance, a major mechanism responsible of the failure of some anticancer drugs. Hence, doxorubicin can more efficiently exert its cytotoxic effect when encapsulated in said nanoparticles.

Moreover, when encapsulated in the form of nanoparticles, doxorubicin can specifically reach the liver, its therapeutic target in HCC.

A Phase II clinical trial has been completed with Livatag® in patients suffering from HCC. Additionally, an international open randomized Phase III clinical trial is underway, aiming to recruit 390 patients with advanced stage HCC and to test Livatag® after failure or intolerance to sorafenib.

Tensioactive agents, such as surfactants, can often impart important and unexpected properties to pharmaceutical compositions. One class of tensioactive agents are the poloxamers, which are synthetic block copolymers of poly(ethylene oxide) and poly(propylene oxide) of the formula:

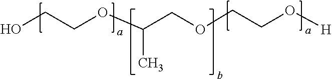

with a and b representing, respectively, the number of ethylene oxide (EO) and propylene oxide (PO) units (also known as oxyethylene and oxypropylene, respectively), and where a is generally between 2 and 130 and b is generally between 15 and 70.

Poloxamers are available in several types, depending on the content of EO and PO (i.e., and thus the a and b values), with various molecular weights and weight percentages of ethylene oxide to propylene oxide (EO/PO).

One particular poloxamer is poloxamer 188. According to the US Pharmacopeia, poloxamer 188 is solid and has an average molecular weight between 7680 and 9510 Da and an average EO weight percentage of 81.8±1.9, i.e., an EO weight percentage of between 79.9 and 83.7%. Poloxamer 188 is commercially available from BASF under the trade names Kolliphor® P 188, Pluronic® F68, and, in the past, Lutrol® F68. According to the BASF manufacturer's data sheets, these products have an EO weight percentage that varies between 79.9 and 83.8% and is typically 81.8%.

In the course of further industrial scale up production of nanoparticle chemotherapeutic drug candidates, it is essential to achieve reproducible nanoparticles with particular specifications, for example, to comply with specific manufacturing and therapeutic requirements and especially regulatory requirements. Among the several parameters to ensure regulatory conformity of said nanoparticles, nanoparticle size is one of the most important.

Indeed, it is generally known that the size of chemotherapeutic agent-loaded nanoparticles is to be such that the nanoparticles are not too small (to avoid elimination by the kidneys, which would result in loss of therapeutic effect) and not too large (to avoid blocking or obstructing blood vessels, which would lead to vessel embolization and thus toxicity).

As a result, it has been determined in the art that chemotherapeutic agent-loaded nanoparticles, such as nanoparticles loaded with doxorubicin, should generally have an average diameter between about 100 and about 300 nm (Hillaireau et al., Cellular and Molecular Life Sciences, 2009, 66:2873-2896). It is essential that this size can be controlled and reproducibly achieved during industrial, large scale, batch manufacturing.

However, known processes for making chemotherapeutic agent-loaded nanoparticles have faced reproducibility problems as the size has not been reliably controlled. Indeed, nanoparticle production batches have been observed to frequently fall outside the desired 100 to 300 nm average diameter for unknown reasons causing such non-compliant batches to be discarded.

WO99/43359 and WO2012/131018 teach that the size of the nanoparticles prepared therein is essentially related to the concentration of cyclodextrin or the type of cyclodextrin. However, contrary to these teachings of WO99/43359 and WO2012/131018, the present application surprisingly shows that poloxamer 188 is responsible for the variation of the size of chemotherapeutic agent-loaded nanoparticles and that known sources of poloxamer 188, which have varying weight percentage of EO, frequently do not lead to nanoparticles with the desired 100-300 nm average diameter.

To solve this problem, the present application surprisingly shows that the EO weight percentage of poloxamer 188 must be within a particular range to achieve the desired 100-300 nm average diameter of nanoparticles.

Further, and contrary to the general expectation in the art, the present application also shows that the molecular weight of poloxamer 188 does not necessarily directly correlate to the size of the nanoparticles, e.g., an increase of the poloxamer 188 molecular weight does not necessarily lead to an increase of the size of the nanoparticles.

### SUMMARY

According to a first object, the present disclosure concerns a nanoparticle or nanoparticles comprising (i) at least one therapeutically active ingredient, (ii) a poly(alkylcyanoacrylate), (iii) one or more cyclodextrin(s), and (iv) poloxamer 188, wherein said poloxamer 188 has an ethylene oxide (EO) weight percentage between 79.9 and 81.5%.

According to a specific embodiment, the EO weight percentage is between 79.9 and 81.0%. According to another embodiment, the EO weight percentage is between 79.9 and 80.8%. According to a further embodiment, the EO weight percentage is between 80.5 and 80.8%.

According to an embodiment, the therapeutically active ingredient is a chemotherapeutic agent, such as an anthracycline, such as doxorubicin or a pharmaceutically acceptable salt thereof.

According to an embodiment, the nanoparticles have an average diameter between about 100 and about 300 nm. such as between about 150 and 300 nm, such as between about 200 and 300 nm. In some embodiments, the nanoparticles are in a dispersion.

### DETAILED DESCRIPTION

According to a first object, the present disclosure concerns a nanoparticle or nanoparticles comprising (i) at least one therapeutically active ingredient, (ii) a poly(alkylcyanoacrylate), (iii) one or more cyclodextrin(s), and (iv) poloxamer 188, wherein said poloxamer 188 has an ethylene oxide (EO) weight percentage between 79.9 and 81.5%.

### Active Ingredient

Examples of therapeutically active ingredients that may be used with the nanoparticles described herein include anticancer agents, antivirals, antibiotics, proteins, polypeptides, polynucleotides, antisense nucleotides, vaccinating substances, immunomodulators, steroids, analgesics, antimorphinics, or antifungals. Among the anticancer agents, chemotherapeutic agents are used in particular embodiments. A preferred chemotherapeutic agent is doxorubicin or a pharmaceutically acceptable salt thereof.

Chemotherapeutic agents can be defined as cytotoxic drugs to treat cancer. Broadly, most chemotherapeutic agents work by impairing mitosis (cell division) or DNA synthesis, effectively targeting fast-dividing cells. As these drugs cause damage to cells they are termed “cytotoxic.”

According to the present description, chemotherapeutic agents can include (i) anthracyclines, (ii) topoisomerase inhibitors, (iii) spindle poison plant alkaloids, (iv) alkylating agents, (v) anti-metabolites, and (vi) other chemotherapeutic agents:

(i) Anthracyclines

Anthracyclines (or anthracycline antibiotics) may be derived from Streptomyces bacteria and may be from natural, synthetic, or semi-synthetic processes. These compounds are used to treat a wide range of cancers, including leukemias, lymphomas, and breast, uterine, ovarian, and lung cancers.

Anthracyclines have three mechanisms of action:
Inhibition of DNA and RNA synthesis by intercalating between base pairs of the DNA/RNA strand, thus preventing the replication of rapidly-growing cancer cells.
Inhibition topoiosomerase II enzyme, preventing the relaxing of supercoiled DNA and thus blocking DNA transcription and replication.
Creation of iron-mediated free oxygen radicals that damage the DNA and cell membranes.

Some non-limiting examples of anthracyclines for use herein are doxorubicin daunorubicin, epirubicin, idarubicin, valrubicin, and pharmaceutically acceptable salts thereof.

(ii) Topoisomerase Inhibitors

Topoisomerases are essential enzymes that maintain the topology of DNA Inhibition of type I or type II topoisomerases interferes with both transcription and replication of DNA by upsetting proper DNA supercoiling.

Some type I topoisomerase inhibitors for use herein include camptothecins derivatives. Camptothecin derivatives refer to camptothecin analogs such as irinotecan, topotecan, hexatecan, silatecan, lutortecan, karenitecin (BNP1350), gimatecan (ST1481), belotecan (CKD602), and pharmaceutically acceptable salts thereof. Irinotecan, its active metabolite SN38, topotecan, and pharmaceutically acceptable salts thereof are used in particular embodiments. Irinotecan and pharmaceutically acceptable salts thereof are used in further embodiments.

Examples of type II topoisomerase inhibitors for use herein include amsacrine, etoposide, etoposide phosphate, teniposide, and pharmaceutically acceptable salts thereof. These are semi-synthetic derivatives of epipodophyllotoxins, alkaloids naturally occurring in the root of American Mayapple (Podophyllum peltatum).

(iii) Spindle Poison Plant Alkaloids

These alkaloids are derived from plants and block cell division by preventing microtubule function, essential for cell division.

Examples of spindle poison plant alkaloids for use herein are vinca alkaloids (such as vinblastine, vincristine, vindesine, vinorelbine, vinpocetine, and pharmaceutically acceptable salts thereof) and taxanes. Taxanes include paclitaxel and docetaxel and their pharmaceutically acceptable salts. Paclitaxel was originally derived from the Pacific yew tree. Docetaxel is a semi-synthetic analogue of paclitaxel.

In contrast to the taxanes, the vinca alkaloids destroy mitotic spindles. Both, taxanes and vinca alkaloids are therefore named spindle poisons or mitosis poisons, but they act in different ways.

(iv) Alkylating Agents

Alkylating agents are so named because of their ability to add alkyl groups to many electronegative groups under conditions present in cells. They impair cell function by forming covalent bonds with the amino, carboxyl, sulfhydryl, and phosphate groups in biologically important molecules. Noteworthy, their cytotoxicity is thought to result from inhibition of DNA synthesis.

Examples of alkylating agents for use herein include platinum compounds, such as oxaliplatin, cisplatin, carboplatin, and pharmaceutically acceptable salts thereof. Other examples of alkylating agents for use herein are mechlorethamine, cyclophosphamide, chlorambucil, ifosfamide, pharmaceutically acceptable salts thereof.

(v) Anti-Metabolites

An anti-metabolite is a chemical that inhibits the use of a metabolite, which is part of normal metabolism. Such substances are often similar in structure to the metabolite that they interfere with. The presence of anti-metabolites alters cell growth and cell division. Examples of anti-metabolites for use herein include purine or pyrimidine analogues and antifolates.

Purine or pyrimidine analogues prevent the incorporation of nucleotides into DNA, stopping DNA synthesis and thus cell division. They also affect RNA synthesis. Examples of purine analogues for use herein include azathioprine, mercaptopurine, thioguanine, fludarabine, pentostatin, cladribine, and pharmaceutically acceptable salts thereof. Examples of pyrimidine analogues for use herein include 5-fluorouracil (5FU), which inhibits thymidylate synthase, floxuridine (FUDR), cytosine arabinoside (Cytarabine), and pharmaceutically acceptable salts thereof.

Antifolates are drugs which impair the function of folic acids. Many are used in cancer chemotherapy, and some are used as antibiotics or antiprotozoal agents. An example for use herein is methotrexate and pharmaceutically acceptable salts thereof. This is a folic acid analogue, and owing to structural similarity with it binds and inhibits the enzyme dihydrofolate reductase (DHFR), and thus prevents the formation of tetrahydrofolate. Tetrahydrofolate is essential for purine and pyrimidine synthesis, and this leads to inhibited production of DNA, RNA and proteins (as tetrahydrofolate is also involved in the synthesis of amino acids serine and methionine). Other examples of antifolates for use herein include trimethoprim, raltitrexed, pyrimethamine, pemetrexed, and pharmaceutically acceptable salts thereof.

(vi) Other Chemotherapeutic Agents

The examples of chemotherapeutic agents above are not limiting and other chemotherapeutic agents can be used herein, including ellipticine and harmine and pharmaceutically acceptable salts thereof.

According to certain embodiments, the therapeutically active ingredient is an anticancer agent, such as a chemotherapeutic agent, such as an anthracycline, such as doxorubicin or a pharmaceutically acceptable salt thereof.

In certain embodiments, the active ingredient, such as a chemotherapeutic agent, such as an anthracycline (e.g., doxorubicin or a pharmaceutically acceptable salt thereof), is present in the nanoparticles at a concentration from about 0.01 to about 200 mg/g of the nanoparticles, preferably from about 1 to about 50 mg/g of the nanoparticles. For example, in some embodiments, the active ingredient, such as a chemotherapeutic agent, such as an anthracycline (e.g., doxorubicin or a pharmaceutically acceptable salt thereof), is present in the nanoparticles at a concentration from about 0.01, 0.02, 0.05, 0.08, 0.1, 0.2, 0.5, 0.8, 1, 2, or 5 to about 10, 25, 35, 50, 75, 100, 125, 150, 175, or 200 mg/g of the nanoparticles;

such as from about 0.01 to about 10 mg/g, from about 0.01 to about 25 mg/g, from about 0.01 to about 50 mg/g, from about 0.01 to about 100 mg/g, from about 0.01 to about 150 mg/g; from about 0.01 to about 175 mg/g;

such as from about 0.05 to about 10 mg/g, from about 0.05 to about 25 mg/g, from about 0.05 to about 50 mg/g, from about 0.05 to about 100 mg/g, from about 0.05 to about 150 mg/g, from about 0.05 to about 200 mg/g;

such as from about 0.1 to about 10 mg/g, from about 0.1 to about 25 mg/g, from about 0.1 to about 50 mg/g, from about 0.1 to about 100 mg/g, from about 0.1 to about 150 mg/g, from about 0.1 to about 200 mg/g;

such as from about 0.5 to about 10 mg/g, from about 0.5 to about 25 mg/g, from about 0.5 to about 50 mg/g, from about 0.5 to about 100 mg/g, from about 0.5 to about 150 mg/g, from about 0.5 to about 200 mg/g;

such as from about 1 to about 10 mg/g, from about 1 to about 25 mg/g, from about 1 to about 50 mg/g, from about 1 to about 100 mg/g, from about 1 to about 150 mg/g, from about 1 to about 200 mg/g;

such as from about 2 to about 10 mg/g, from about 2 to about 25 mg/g, from about 2 to about 50 mg/g, from about 2 to about 100 mg/g, from about 2 to about 150 mg/g, from about 2 to about 200 mg/g; or

such as from about 5 to about 10 mg/g, from about 5 to about 25 mg/g, from about 5 to about 50 mg/g, from about 5 to about 100 mg/g, from about 5 to about 150 mg/g, from about 5 to about 200 mg/g.

### Poly(Alkylcyanoacrylate)

The poly(alkylcyanoacrylate) may be linear or branched, preferably branched. The alkyl group of the poly(alkylcyanoacrylate) may be linear or branched, preferably branched. In a particular embodiment, the poly(alkylcyanoacrylate) is a poly(C1-C12) alkylcyanoacrylate, preferably a poly(C4-C10) alkylcyanoacrylate, more preferably a poly(C6-C8) alkylcyanoacrylate. In a preferred embodiment, the poly(alkylcyanoacrylate) is a polyisohexylcyanoacrylate (PIHCA). In another preferred embodiment, the poly(alkylcyanoacrylate) is a polyethylbutylcyanoacrylate (PEBCA). The monomer corresponding to the latter polymer is available, for instance under the trademark Monorex® by Onxeo (France).

In certain embodiments, the poly(alkylcyanoacrylate) is present in the nanoparticles from about 0.5% to about 25% by weight of the nanoparticles, preferably from about 5% to about 15% by weight of the nanoparticles. For example, in some embodiments, the poly(alkylcyanoacrylate) is present in the nanoparticles from about 0.5, 1, 2, 5, 7, or 9% to about 10, 12, 15, 17, 20, 22, or 25%,

such as from about 0.5% to about 10%, from about 0.5% to about 12%, from about 0.5% to about 15%, from about 0.5% to about 17%, from about 0.5% to about 20%, from about 0.5% to about 22%;

such as from about 1% to about 10%, from about 1% to about 12%, from about 1% to about 15%, from about 1% to about 17%, from about 1% to about 20%, from about 1% to about 25%;

such as from about 2% to about 10%, from about 2% to about 12%, from about 2% to about 15%, from about 2% to about 17%, from about 2% to about 20%, from about 2% to about 25%;

such as from about 5% to about 10%, from about 5% to about 12%, from about 5% to about 15%, from about 5% to about 17%, from about 5% to about 20%, from about 5% to about 25%;

such as from about 7% to about 10%, from about 7% to about 12%, from about 7% to about 15%, from about 7% to about 17%, from about 7% to about 20%, from about 7% to about 25%; or

such as from about 9% to about 10%, from about 9% to about 12%, from about 9% to about 15%, from about 9% to about 17%, from about 9% to about 20%, from about 9% to about 25%.

### Cyclodextrin

The cyclodextrin may be neutral or charged, native (cyclodextrins α, β, γ, δ, ε), branched or polymerized, or even chemically modified, for example, by substitution of one or more hydroxy groups by groups such as alkyls, aryls, arylalkyls, glycosidics, or by etherification, esterification with alcohols or aliphatic acids. Among the above groups, particular preference is given to those chosen from the group consisting of hydroxypropyl, methyl and sulfobutylether groups, and mixtures thereof. In a preferred embodiment, the cyclodextrin is selected from the group consisting of hydroxypropyl-beta-cyclodextrin and/or randomly methylated-beta cyclodextrin, and mixtures thereof. In a particular embodiment, the cyclodextrin is hydroxypropyl-beta-cyclodextrin.

In certain embodiments, cyclodextrin is present in the nanoparticles from about 0.1% to about 70% by weight of the nanoparticles, preferably from about 1% to about 30%, more preferably from about 5% to about 20%. For example, in some embodiments, the cyclodextrin is present in the nanoparticles from about 0.1, 0.2, 0.5, 0.8, 1, 2, 5, 7, 10, or 12% to about 13, 15, 20, 25, 35, 50, 60, or 70% by weight of the nanoparticles,

such as from about 0.1% to about 15%, from about 0.1% to about 20%, from about 0.1% to about 25%, from about 0.1% to about 35%, from about 0.1% to about 50%, from about 0.1% to about 60%;

such as from about 0.5% to about 15%, from about 0.5% to about 20%, from about 0.5% to about 25%, from about 0.5% to about 35%, from about 0.5% to about 50%, from about 0.5% to about 70%;

such as from about 1% to about 15%, from about 1% to about 20%, from about 1% to about 25%, from about 1% to about 35%, from about 1% to about 50%, from about 1% to about 70%;

such as from about 2% to about 15%, from about 2% to about 20%, from about 2% to about 25%, from about 2% to about 35%, from about 2% to about 50%, from about 2% to about 70%;

such as from about 5% to about 15%, from about 5% to about 20%, from about 5% to about 25%, from about 5% to about 35%, from about 5% to about 50%, from about 5% to about 70%; or

such as from about 10% to about 15%, from about 10% to about 20%, from about 10% to about 25%, from about 10% to about 35%, from about 10% to about 50%, from about 10% to about 70%.

### Poloxamer

According to some embodiments, the EO weight percentage of the poloxamer 188 is between 79.9% and 81.5%, such as between 79.9, 80.0, 80.1, 80.2, 80.3, 80.4, 80.5, or 80.6% and 80.7, 80.8, 80.9, 81.0, 81.1, 81.2, 81.3, 81.4, or 81.5%, for example

between 79.9% and 81.4%, 79.9% and 81.3%, 79.9% and 81.2%, 79.9% and 81.1%, 79.9% and 81.0%, 79.9% and 80.9%, 79.9% and 80.8%, 79.9% and 80.7%, 79.9% and 80.6%, or 79.9% and 80.5%; or

between 80.0% and 81.4%, 80.0% and 81.3%, 80.0% and 81.2%, 80.0% and 81.1%, 80.0% and 81.0%, 80.0% and 80.9%, 80.0% and 80.8%, 80.0% and 80.7%, 80.0% and 80.6%, or 80.0% and 80.5%; or

between 80.2% and 81.4%, 80.2% and 81.3%, 80.2% and 81.2%, 80.2% and 81.1%, 80.2% and 81.0%, 80.2% and 80.9%, 80.2% and 80.8%, 80.2% and 80.7%, 80.2% and 80.6%, or 80.2% and 80.5%; or

between 80.3% and 81.4%, 80.3% and 81.3%, 80.3% and 81.2%, 80.3% and 81.1%, 80.3% and 81.0%, 80.3% and 80.9%, 80.3% and 80.8%, 80.3% and 80.7%, 80.3% and 80.6%, or 80.3% and 80.5%; or

between 80.5% and 81.4%, 80.5% and 81.3%, 80.5% and 81.2%, 80.5% and 81.1%, 80.5% and 81.0%, 80.5% and 80.9%, 80.5% and 80.8%, 80.5% and 80.7%, or 80.5% and 80.6%.

According to a specific embodiment, the EO weight percentage of the poloxamer 188 is between 79.9 and 81.0%. According to another embodiment, the EO weight percentage is between 79.9 and 80.8%. According to a further embodiment, the EO weight percentage is between 80.5 and 80.8%.

According to another specific embodiment, the EO weight percentage of the poloxamer 188 is between 80.0 and 81.5%. According to another embodiment, the EO weight percentage is between 80.0 and 81.0%. According to a further embodiment, the EO weight percentage is between 80.0 and 80.8%.

The EO weight percentage as used herein refers to the content of ethylene oxide in weight with respect to the total weight of poloxamer 188 in a given sample, such as a given sample of nanoparticles or an individual nanoparticle. The EO weight percentage may be obtained by standard procedures, such as nuclear magnetic resonance (NMR), such as those disclosed in the US pharmacopeia and reported in the examples described herein.

### Nanoparticle Size

According to an embodiment, the nanoparticles described herein have an average diameter between about 100 nm and about 300 nm, such as between about 100, 125, 150, 175, 200, or 225 nm and about 250, 275, or 300 nm, for example

between about 100 nm and about 250 nm, between about 100 nm and about 275 nm, or between about 100 nm and about 300 nm,

between about 125 nm and about 250 nm, between about 125 nm and about 275 nm, or between about 125 nm and about 300 nm,

between about 150 nm and about 250 nm, between about 150 nm and about 275 nm, or between about 150 nm and about 300 nm,

between about 175 nm and about 250 nm, between about 175 nm and about 275 nm, or between about 175 nm and about 300 nm,

between about 200 nm and about 250 nm, between about 200 nm and about 275 nm, or between about 200 nm and about 300 nm, or

between about 225 nm and about 250 nm, between about 225 nm and about 275 nm, or between about 225 nm and about 300 nm.

In particular embodiments, the nanoparticles described herein have an average diameter between about 150 and about 300 nm, such as between about 200 and about 300 nm, such as between about 200 and about 275 nm.

The average diameter of nanoparticles as used herein refers to the average value of the diameter of nanoparticles in a given sample. The average diameter can be obtained by application of known procedures, such as Dynamic Light Scattering and reported in the examples described herein.

### DEFINITIONS AND FURTHER EMBODIMENTS

As used herein, the term “pharmaceutically acceptable” refers to those compounds, materials, excipients, compositions or dosage forms which are, within the scope of sound medical judgment, suitable for contact with the tissues of human beings and animals without excessive toxicity, irritation, allergic response or other problem complications commensurate with a reasonable benefit/risk ratio.

As used herein, “pharmaceutically acceptable salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, tartaric, citric, methanesulfonic, benzenesulfonic, glucoronic, glutamic, benzoic, salicylic, toluenesulfonic, oxalic, fumaric, maleic, lactic and the like. Further addition salts include ammonium salts such as tromethamine, meglumine or epolamine, metal salts such as sodium, potassium, calcium, zinc or magnesium. For instance, a suitable salt of doxorubicin is doxorubicin hydrochloride.

As used in this specification, the term “about” refers to a range of values ±10% of the specified value. For instance, “about 1” means from 0.9 to 1.1 when 10% is considered and from 0.95 to 1.05 when 5% is considered. Where “about” is used in connection with numeric ranges, for example “about 1 to about 3”, or “between about one and about three”, preferably the definition of “about” given above for a number is applied to each number defining the start and the end of a range separately. Preferably, where “about” is used in connection with any numerical values, the “about” can be deleted.

In certain embodiments, the nanoparticles disclosed and prepared herein are chemically “homogenous,” meaning that they have the same or substantially the same chemical components, such as (i) the least one therapeutically active ingredient, (ii) the poly(alkylcyanoacrylate), (iii) the one or more cyclodextrin(s), and (iv) the poloxamer 188 (e.g., when measured based on EO weight percentage). Individual homogenous nanoparticles can still have different sizes from each other, but a sample of homogeneous nanoparticles has an average diameter as discussed herein.

In a particular embodiment, the nanoparticle or nanoparticles comprise doxorubicin or a pharmaceutically acceptable salt thereof, at least one poly(C1-C12 alkylcyanoacrylate), preferably a polyethylbutylcyanoacrylate (PEBCA), at least one cyclodextrin, preferably selected from the group consisting of hydroxypropyl-beta-cyclodextrin and randomly methylated-beta cyclodextrin, and mixtures thereof and poloxamer 188, wherein said poloxamer 188 has an ethylene oxide (EO) weight percentage between 79.9 and 81.5%.

In a particularly preferred embodiment, the nanoparticle or nanoparticles comprise doxorubicin or a pharmaceutically acceptable salt thereof, a polyethylbutylcyanoacrylate, a hydroxypropyl-beta-cyclodextrin and poloxamer 188, wherein said poloxamer 188 has an ethylene oxide (EO) weight percentage between 79.9 and 81.5%.

In some embodiments, the proportion of the active ingredient and the proportion of cyclodextrin in the nanoparticles are generally independent from one another.

In certain embodiments, the nanoparticles described herein comprise:
doxorubicin or a pharmaceutically acceptable salt thereof at a concentration from 0.01 to 200 mg/g of nanoparticles, and/or
from 0.1 to 70% w/w of said at least one cyclodextrin; and
from 1 to 25% w/w of said poly(alkylcyanoacrylate).

In a particular embodiment, the nanoparticles described herein may comprise more than one active ingredient, such doxorubicin or a pharmaceutically acceptable salt thereof in combination with another chemotherapeutic agent.

According to a second object, the present disclosure also concerns a process for preparing nanoparticles loaded with at least one active ingredient, said process comprising:
a) mixing at least one active ingredient (such as doxorubicin or a pharmaceutically acceptable salt thereof) with at least one cyclodextrin and poloxamer 188, wherein said poloxamer 188 has an ethylene oxide (EO) weight percentage between 79.9 and 81.5%, in a polymerization medium;
b) adding an alkylcyanoacrylate monomer to said mixture; and
c) conducting polymerization, optionally in the presence of one or more surfactant and/or stabilizing agents,
wherein said at least one active ingredient, cyclodextrin, poloxamer 188, and alkylcyanoacrylate monomer are defined as above.

The present disclosure also includes nanoparticles prepared by the processes described herein.

The nanoparticles of the present disclosure may be prepared by application or adaptation of any method known by the skilled person, within the scope of the processes of the present disclosure. Such methods are disclosed, for example, in WO99/43359.

In particular, the nanoparticles may be prepared by a method comprising:
a) preparing, in aqueous acid, a complex of at least one active ingredient (such as doxorubicin or a pharmaceutically acceptable salt thereof) and at least one cyclodextrin and mixing with poloxamer 188, wherein said poloxamer 188 has an EO weight percentage between 79.9 and 81.5%;
b) gradually adding an alkylcyanoacrylate monomer, preferably an isohexylcyanoacrylate monomer or a 2-ethylbutylcyanoacrylate monomer, to the mixture obtained at step (a); and
c) conducting polymerization of the monomer, optionally in the presence of one or more surfactant and/or stabilizing agents,
wherein said at least one active ingredient, cyclodextrin, poloxamer 188, and alkylcyanoacrylate monomer are defined as above.

In certain embodiments, the above processes comprise the step of selecting poloxamer 188 with an ethylene oxide (EO) weight percentage between 79.9 and 81.5% (or any of the other EO weight percentage ranges recited for the nanoparticles herein) prior to mixing said poloxamer 188 with the at least one active ingredient and the at least one cyclodextrin.

Preferably, the polymerization of the processes is anionic but may also be inducible by other agents, in particular by photochemical agents. According to an embodiment, the polymerization is carried out in an aqueous medium at a pH between about 3 and about 4.

Surfactant agents include dextran (such as dextran 70,000) or other non-ionic surfactant agents (such as polysorbate and sorbitan esters).

According to an embodiment of the processes of the present disclosure, the EO weight percentage of poloxamer 188 is between 79.9 and 81.0%. According to another embodiment, the EO weight percentage is between 80.0 and 81.0%. According to another embodiment, the EO weight percentage is between 80.0 and 80.8%. According to a further embodiment, the EO weight percentage is between 80.5 and 80.8%. Additional EO weight percentages for the processes of the disclosure are described above along with further specifications for the active ingredient(s), the cyclodextrin(s), the alkylcyanoacrylate monomer, and resulting polymer, the poloxamer 188, and the nanoparticles prepared by the processes.

According to an embodiment, the processes further comprise recovering the nanoparticles from the polymerization medium, for example by filtering the nanoparticles.

According to an embodiment, the processes further comprise suspending or resuspending the nanoparticles into a dispersion medium.

According to an embodiment, the polymerization and/or dispersion medium is an aqueous medium.

The present disclosure also concerns nanoparticles obtainable by the processes of the present disclosure. According to an embodiment, the nanoparticles have an average diameter between 100 and 300 nm, such as between about 150 and 300 nm, such as between about 200 and 300 nm. Additional average diameter ranges for the nanoparticles are discussed above.

The present disclosure also concerns a dispersion comprising said nanoparticles in a dispersion medium, such as an aqueous medium.

A “dispersion” as used herein refers to a mixture in which the nanoparticles are dispersed throughout a continuous phase, i.e., the dispersion medium. Said dispersions include suspensions, colloids, and solutions.

The term “dispersion medium” as used herein refers to the continuous phase of the dispersions described herein. It may be an aqueous or a nonaqueous medium. It may be also the polymerization medium in which the nanoparticles are synthesized, or it may be a different medium in which the nanoparticles are resuspended following synthesis and filtration from the polymerization medium.

According to an embodiment, the dispersion is an aqueous suspension, having a pH between about 0.5 and about 5, such as between about 1.5 and about 5, such as between about 2.5 and about 4.5, more particularly between about 3 and about 4. This pH may be achieved by adding a suitable acid, base, or buffer such as citric acid to the suspension medium.

The nanoparticles as described above can be administered in the form of a pharmaceutical composition comprising said nanoparticles and at least one pharmaceutically acceptable excipient, such as a suitable solution for intravenous, intra-arterial, and intra-tumoral administration.

According to a further object, the present disclosure also concerns a pharmaceutical composition comprising the nanoparticles described herein and at least one pharmaceutically acceptable excipient.

The disclosure also concerns a method of treating cancer, such as a liver cancer, such as HCC, comprising administering the nanoparticles described herein to a patient in need thereof, such as a therapeutically effective amount of the nanoparticles described herein to a patient in need thereof.

The nanoparticles for use in the treatment of cancer, such as a liver cancer, such as HCC, are another object of the present disclosure.

The use of the nanoparticles for the manufacture of a medication dedicated to the treatment of cancer, such as a liver cancer, such as HCC, is also another object of the present disclosure.

The pharmaceutical composition comprising said nanoparticles may be formulated in accordance with standard pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art. In particular, possible pharmaceutical compositions include those suitable for intravenous, intra-arterial, and intra-tumoral administration. For these formulations, conventional excipients can generally be used according to techniques well known by those skilled in the art. Such compositions for parenteral administration are generally physiologically compatible sterile solutions or suspensions which can optionally be prepared immediately before use from solid or lyophilized form. Adjuvants such as a local anesthetic, preservative and buffering agents can be dissolved in the vehicle and a surfactant or wetting agent can be included in the composition to facilitate uniform distribution of the nanoparticles.

In addition to nanoparticles as described above, the pharmaceutical composition may further comprise at least one additional active substance, such as another chemotherapeutic agent included or not included in nanoparticles.

The nanoparticles used in the present disclosure may be administered to a patient in need thereof to provide a therapeutically effective amount of the at least one active ingredient, such as a chemotherapeutic agent, such as doxorubicin or a pharmaceutically acceptable salt thereof.

As used herein, the term “patient” refers to either an animal, such as a valuable animal for breeding, company or preservation purposes, or preferably a human (child or adult), which is afflicted with, or has the potential to be afflicted cancer.

As used herein, a “therapeutically effective amount” refers to an amount of a compound which is effective in preventing, reducing, eliminating, treating, or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment. The amount of nanoparticles to be administered generally is determined by standard procedures well known by those of ordinary skill in the art. In particular, physiological data of the patient (e.g. age, size, and weight), the type and localization of cancer, the nature of the chemotherapeutic agent are taken into account to determine the appropriate dosage. In a particular embodiment, the nanoparticles are administered in an amount providing a dosage of doxorubicin or a pharmaceutically acceptable salt thereof from about 10 to about 75 mg/m², preferably from about 10 to about 60 mg/m², preferably from about 10 to about 45 mg/m², more preferably from about 10 to about 30 mg/m², from about 20 to about 30 mg/m². More preferably, the dosage of doxorubicin or a pharmaceutically acceptable salt thereof may be about 20 mg/m² or 30 mg/m². Here, “m²” refers to the body surface area of a patient, which can be calculated by the skilled person from the patient's body weight and height (e.g., body weight of about 65 kg corresponds to a body surface of about 1.8 mg/m²).

The identification of those subjects who are in need of treatment of herein-described diseases and conditions is well within the ability and knowledge of one skilled in the art. A clinician skilled in the art can readily identify, by the use of clinical tests, physical examination and medical/family history, those subjects who are in need of such treatment.

Upon considering the present disclosure, a therapeutically effective amount can be readily determined by the attending diagnostician, as one skilled in the art, by the use of conventional techniques and by observing results obtained under analogous circumstances. In determining the therapeutically effective amount, a number of factors are considered by the attending diagnostician, including, but not limited to: the species of subject; its size, age, and general health; the specific disease involved; the degree of involvement or the severity of the disease; the response of the individual subject; the mode of administration; the bioavailability characteristic of the preparation administered; the dose regimen selected; the use of concomitant medication; and other relevant circumstances.

The amount of nanoparticles, which is required to achieve the desired biological effect will vary depending upon a number of factors, including the dosage of the drug to be administered, the type of disease, the diseased state of the patient, and the route of administration.

The preferred dosage of drug to be administered is likely to depend on such variables as the type and extent of progression of the disease or disorder, the overall health status of the particular patient and its route of administration.

The nanoparticles can be formulated into pharmaceutical compositions by admixture with one or more pharmaceutically acceptable excipients. Such compositions may be prepared for use in oral administration, particularly in the form of tablets or capsules; or parenteral administration, particularly in the form of liquid solutions, suspensions, or emulsions; or intranasally, particularly in the form of powders, nasal drops, or aerosols; or dermally, for example, topically or via trans-dermal patches. Parenteral administration is preferred and includes:
intravenous (into a vein),
intra-arterial (into an artery),
intraosseous infusion (into the bone marrow),
intra-muscular,
intracerebral (into the brain parenchyma),
intracerebroventricular (into cerebral ventricular system),
intrathecal (an injection into the spinal canal), and
subcutaneous (under the skin).

Liquid preparations for administration include sterile aqueous or nonaqueous solutions, dispersions, such as suspensions and emulsions. The liquid compositions may also include binders, buffers, and preservatives as well as chelating, sweetening, flavoring, and coloring agents and the like. Suitable nonaqueous solvents include alcohols, propylene glycol, polyethylene glycol, vegetable oils such as olive oil, and organic esters such as ethyl oleate. Aqueous carriers include mixtures of alcohols and water, buffered media, and saline. In particular, biocompatible, biodegradable lactide polymer, lactide/glycolide copolymer, or polyoxyethylene-polyoxypropylene copolymers may be useful excipients to control the release of the active compounds.

Intravenous vehicles can include fluid and nutrient replenishers, electrolyte replenishers, such as those based on Ringer's dextrose, and the like. Other potentially useful parenteral delivery systems for these active compounds include ethylene-vinyl acetate copolymer particles, osmotic pumps, implantable infusion systems, and liposomes.

As used herein, the term “intravenous administration” (“IV”) refers to the infusion of liquid substances directly into a vein. This term refers to any type of intravenous access devices. In particular, this term refers to hypodermic needle. It is the simplest form of intravenous access by passing a hollow needle through the skin directly into the vein. This needle can be connected directly to a syringe or may be connected to a length of tubing and thence whichever collection or infusion system is desired.

Administration methods, such as intraventricular and intraarterial infusion are described in WO2012/131018 and its US counterparts (e.g., U.S. Published Patent Application No. 2014/0024610), which are incorporated by reference herein in their entirety.

As used herein, the term “cancer” refers to the presence of cells possessing characteristics typical of cancer-causing cells, such as uncontrolled proliferation, immortality, metastatic potential, rapid growth and proliferation rate, and certain characteristic morphological features. This term refers to any type of malignancy (primary or metastases). In particular, this term refers to any malignant proliferative cell disorders such as solid tumor or hematopoietic tumor, including carcinoma, sarcoma, lymphoma, stem cell tumor, and blastoma. In certain embodiments, the cancer treated with the nanoparticles described herein is selected from the group consisting of hepatic cancer, in particular hepatocellular carcinoma, acute lymphoblastic leukemia, acute myeloblastic leukemia, chronic myelogenous leukemia, Hodgkin's disease, diffuse large B-cell lymphoma, lung cancer (such as small cell lung cancer), colorectal cancer, pancreas cancer, breast cancer, ovary cancer, uterine cancer, cervix cancer, head and neck cancer, brain cancer, blade cancer, multiple myeloma, neuroblastoma, Edwing's sarcoma, osteosarcoma, soft tissue sarcoma, thyroid cancer, prostate cancer, stomach cancer, nephroblastoma, Kaposi's sarcoma, and non-Hodgkin's lymphoma. In particular embodiments, the cancer treated with the nanoparticles described herein is hepatic cancer, in particular, hepatocellular carcinoma.

The following examples are given for purposes of illustration and are not intended to be limiting.

### EXAMPLES

### Example 1

### Preparation of Nanoparticles Loaded with Doxorubicin

Livatag® nanoparticles are presented as a sterile lyophilisate for injectable suspension that contains doxorubicin hydrochloride as active ingredient and other excipients including the polymer polyethylbutylcyanoacrylate (PEBCA).

The doxorubicin loaded nanoparticles are obtained by aqueous emulsion polymerization of 2-ethylbutylcyanoacrylate (EBCA) or isohexylcyanoacrylate (IHCA) monomer dropped in the bulk solution containing the active ingredient doxorubicin and the other below excipients (see P. Couvreur, B. Kante, M. Roland, P. Guiot, P. Baudhuin, P. Speiser, J. Pharm. Pharmacol., 1979, 31, 331). At the end of polymerization, a stable suspension of nanoparticles entrapping doxorubicin is obtained. The nanoparticle suspension is then filtered and aseptically filled in glass vials before freeze-drying. For stability purposes, the nanoparticles loaded with doxorubicin freeze-dried product is preferably kept protected from light and humidity and stored in a refrigerator between 2° C. and 8° C.

Raw Materials. For 100 ml of total volume of polymerization media:

| Excipients | Quantity (gram) |
|---|---|
| Poloxamer 188 | 1 |
| Hydroxypropyl-beta cyclodextrin | 0.5 |
| H₂O | qsp 100 ml |
| Anhydrous citric acid (1M) | 0.5 qsp pH = 3 to 4 |

Method for preparing 100 ml of polymerization Medium (pH between 3 and 4):
Dissolution of doxorubicin in water at 10 mg/ml
Addition of poloxamer 188 (concentration between 0.5 and 15%), under stirring and until complete dissolution occurs
Addition of cyclodextrin (concentration between 0.1 and 70%)
Adjustment to the required pH (between 3 and 4) with citric acid 0.1 M

Method for preparing nanoparticles loaded with Doxorubicin:
In a 10 ml flask with magnetic stirring:
Add 5 ml of polymerization media
Add 50 μl of EBCA or IHCA solution gassed with SO2 (density of EBCA=0.980)
Let polymerization occur for 2 h 30 min or longer as needed under magnetic stirring and at room temperature
Filter through 2 μm filter

Example 1 was repeated with various samples of poloxamer 188 having different EO weight percentages (cf. Example 2).

### Example 2

### Assessment of the Nanoparticle Parameters

2.1. The EO weight percentage of poloxamer 188 was obtained according to the following procedure in accordance with the US Pharmacopeia:

Solvent—Use deuterated water or deuterochloroform.

NMR reference—Use sodium 2,2-dimethyl-2-silapentane-5-sulfonate (for deuterated water) or tetramethylsilane (for deuterochloroform).

Test preparation—Dissolve 0.1 g to 0.2 g of poloxamer in deuterated water containing 1% of sodium 2,2-dimethyl-2-silapentane-5-sulfonate to obtain 1 mL of solution, or, if the poloxamer does not dissolve in water, use deuterochloroform containing 1% of tetramethylsilane as the solvent.

Procedure—Transfer 0.5 mL to 1.0 mL of the Test preparation to a standard 5-mm NMR spinning tube, and if deuterochloroform is the solvent, add 1 drop of deuterated water, and shake the tube. Proceed as directed for Relative Method of Quantitation under NMR, using the Test preparation volumes specified here, scanning the region from 0 ppm to 5 ppm, and using the calculation formulas specified here. Record as A1 the average area of the doublet appearing at about 1.08 ppm, representing the methyl groups of the oxypropylene (PO) units, and record as A2 the average area of the composite band from 3.2 ppm to 3.8 ppm, due to the CH2O groups of both the oxyethylene (E0) and oxypropylene units and also the CHO groups of the oxypropylene units, with reference to the sodium 2,2-dimethyl-2-silapentane-5-sulfonate or tetramethylsilane singlet at 0 ppm. Calculate the percentage of oxyethylene, by weight, in the Poloxamer, taken by the formula:

3300α/(33α+58),

in which α is (A2/A1)−1.
pH: between 5.0 and 7.5, in a solution (1 in 40).

2.2. The average diameter of the nanoparticles obtained after conducting the procedure of Example 1 was calculated according to the following procedure:
Place 2.5 μL of nanoparticles suspensions into 3 mL of MilliQ water.
Shake gently to avoid bubbles
Analyze the sample by Dynamic Light Scattering (T° C. 20° C., 3 measurements per preparation, equilibration 120 s)
The results are given in term of mean Z-average of the 3 measurements

2.3. 2 liter formulation. In this experiment, nanoparticles were obtained according to Example 1 following a clarifying filtration.

| | Sample 1 | Sample 2 | Sample 3 |
|---|---|---|---|
| Poloxamer 188 | 9215 | 8436 | 8456 |
| Molecular Weight (g/mol) | | | |
| Poloxamer 188 | 80.5 | 82.5 | 81.0 |
| EO weight (%) | | | |
| Average diameter of the | 269 | 389 | 223 |
| nanoparticles (nm) | | | |

2.4. 24 liter formulation. In this experiment, nanoparticles were obtained according to Example 1 following by a double filtration and lyophilization:

| | Sample 4 | Sample 5 |
|---|---|---|
| Poloxamer 188 | 9429 | 8385 |
| Molecular Weight (g/mol) | | |
| Poloxamer 188 | 82.5 | 80.8 |
| EO weight (%) | | |
| Average diameter of the | 391 | 200 |
| nanoparticles (nm) | | |

The results summarized in Examples 2.3 and 2.4 above show that poloxamer 188 with EO weight percentage between 80.0 and 81.5% consistently leads to nanoparticles of average diameter between 200 and 300 nm, whereas poloxamer 188 with EO weight percentage above 81.5% leads to nanoparticles of average diameter of more than 300 nm. Further, these examples show that the molecular weight of poloxamer 188 does not necessarily directly correlate to the size of the nanoparticles, e.g., an increase of the poloxamer 188 molecular weight does not necessarily lead to an increase in the size of the nanoparticles.

The entire disclosures of all publications and documents cited herein are expressly incorporated herein by reference for all purposes.

1. Nanoparticles comprising:
(i) an active ingredient,
(ii) a poly(alkylcyanoacrylate),
(iii) a cyclodextrin, and
(iv) poloxamer 188,
wherein the poloxamer 188 has an ethylene oxide (EO) weight percentage between 79.9 and 81.5%.

2. The nanoparticles according to claim 1, wherein the poloxamer 188 has an EO weight percentage between 79.9 and 81.0%.

3. The nanoparticles according to claim 1, wherein the poloxamer 188 has an EO weight percentage between 79.9 and 80.8%.

4. The nanoparticles according to claim 1, wherein the poloxamer 188 has an EO weight percentage between 80.5 and 80.8%.

5. The nanoparticles according to claim 1, wherein the active ingredient is a chemotherapeutic agent.

6. The nanoparticles according to claim 5, wherein the active ingredient is an anthracycline.

7. The nanoparticles according to claim 6, wherein the active ingredient is doxorubicin or a pharmaceutically acceptable salt thereof.

8. The nanoparticles according to claim 1, wherein the nanoparticles have an average diameter between about 100 and about 300 nm.

9. The nanoparticles according to claim 1, wherein the nanoparticles have an average diameter between about 150 and about 300 nm.

10. The nanoparticles according to claim 1, wherein the nanoparticles have an average diameter between about 200 and about 300 nm.

11. The nanoparticles according to claim 1, wherein the poly(alkylcyanoacrylate) is polyethylbutylcyanoacrylate (PEBCA).

12. A process for preparing nanoparticles loaded with an active ingredient comprising:
a) mixing the active ingredient with a cyclodextrin and poloxamer 188, wherein the poloxamer 188 has an ethylene oxide (EO) weight percentage between 79.9 and 81.5%;
b) adding an alkylcyanoacrylate monomer to said mixture; and
c) polymerizing the alkylcyanoacrylate monomer in a polymerization medium;
thereby preparing nanoparticles loaded with the active ingredient.

13. The process according to claim 12, which further comprises, prior to step a), selecting poloxamer 188 with an ethylene oxide (EO) weight percentage between 79.9 and 81.5%.

14. The process according to claim 12, wherein the active ingredient is doxorubicin or a pharmaceutically acceptable salt thereof.

15. The process according to claim 12, which further comprises recovering the nanoparticles from the polymerization medium.

16. The process according to claim 15, which further comprises resuspending the nanoparticles into a dispersion medium.

17. The process according to claim 12, wherein the polymerization medium is an aqueous medium.

18. The process according to claim 17, wherein the polymerization is carried out at a pH between about 3 and about 4.

19. Nanoparticles prepared by the process according to claim 12.

20. A dispersion comprising the nanoparticles according to claim 1 in a dispersion medium.

21. A method for treating cancer comprising administering to a patient in need thereof a therapeutically effective amount of the nanoparticles according to claim 1.
